(19) United States
(12) Patent Application Publication
(10) Pub. No.: US 2024/0374208 A1
(43) Pub. Date: Nov. 14, 2024

(54) SYSTEM AND METHOD OF PROGRESSION ASSESSMENT OF A NEUROLOGICAL DISEASE

(71) Applicant: OWLYTICS HEALTHCARE LTD, Bnei Atarot (IL)

(72) Inventors: Gill ZAPHRIR, Bnei Atarot (IL); Nathaniel SHIMONI, Be'er Sheva (IL); Yaron RECHER, Ramat Yishai (IL)

(73) Assignee: OWLYTICS HEALTHCARE LTD, Bnei Atarot (IL)

(21) Appl. No.: 18/657,914

(22) Filed: May 8, 2024

Related U.S. Application Data
(60) Provisional application No. 63/464,707, filed on May 8, 2023.

(51) Int. Cl. A61B 5/00; A61B 5/0205; A61B 5/024; A61B 5/103; A61B 5/11; A61B 5/1455

(52) U.S. Cl. CPC A61B 5/4842; A61B 5/0205; A61B 5/1038; A61B 5/1118; A61B 5/112; A61B 5/14551; A61B 5/6807; A61B 5/681; A61B 5/02405; A61B 2562/0219; A61B 2562/0247

(57) ABSTRACT

Systems and methods of progression (e.g. continuous ongoing and long term) assessment of a neurological disease of a patient, including: receiving, from a device that is wearable by the patient, 3D acceleration data and at least one physiological signal; determining, by a server, with a dedicated deep learning algorithm a progression of a neurological disease of the patient based on the received 3D acceleration data and the at least one physiological signal, wherein determination of the progression of the neurological disease includes: determining a first metric for patient's activity, determining a second metric based on a measured gait speed, and aggregating the first metric and the second metric into a combined approximation of progression of the neurological disease.

### CROSS REFERENCE TO RELATED APPLICATION

This application claims benefit from U.S. Provisional Patent Application No. 63/464,707, filed May 8, 2023, which is incorporate herein by reference.

### FIELD OF THE INVENTION

The present invention relates to wearable technology and digital health. More specifically, the present invention relates to systems and methods for non-invasive tracking of the progression assessment of neurological diseases.

### BACKGROUND

Existing solutions for tracking disease progression are often expensive, time-consuming, and require specialized equipment.

Clinical rating scales can be used as subjective scales to assess the severity of motor and/or non-motor symptoms of neurological diseases. They involve a clinician asking the patient questions or making observations about the patient's symptoms and assigning a score based on their short time-windows observations.

Various imaging techniques, such as magnetic resonance imaging (MRI) and positron emission tomography (PET), can be used to detect structural and functional changes in the brain or nervous system.

Other lab electrophysiological measurements can be used, such as electroencephalography (EEG), which measures the electrical activity of the brain, and electromyography (EMG), which measures the electrical activity of muscles.

Wearable devices, including accelerometers and physiological signals, are non-invasive and portable sensors that can be attached to the patient's body to monitor their movements and gait. These devices are designed to measure changes in acceleration and can provide quantitative information about the patient's physical activity and movement patterns.

In the context of neurological disease, accelerometers can be used to assess gait disturbances, tremors, and other motor symptoms.

Generally, all commercially available solutions require that the patient is monitored within clinics and specialized labs, for instance to evaluate the effectiveness of treatments. These solutions allow for periodic tests only.

### SUMMARY OF THE INVENTION

Non-invasive collection and analysis of patient data may provide a continuous, ongoing (e.g., for years) measurement of gait speed and/or disease progression in a cost-effective, convenient and efficient solution. Some embodiments may use a wearable device which is not harnessed to the center of body mass.

There is thus provided, in accordance with some embodiments of the invention, a method of progression assessment of a neurological disease of a patient, including receiving, from a device that is wearable by the patient, three-dimensional (3D) acceleration data and at least one physiological signal, determining, by a server, with a dedicated deep learning algorithm a progression of a neurological disease of the patient based on the received 3D acceleration data and the at least one physiological signal, wherein determination of the progression of the neurological disease includes: determining a first metric for patient's activity, determining a second metric based on a measured gait speed, and aggregating the first metric and the second metric into a combined approximation of progression of the neurological disease.

In some embodiments, the at least one physiological signal includes information on heart activity with at least one of: heart rate data, heart rate variability data and a lead electrocardiogram (ECG). In some embodiments, the at least one physiological signal includes oxygen saturation with SPO2 measurements. In some embodiments, the 3D acceleration data is sampled with a frequency of at least 25 Hz.

In some embodiments, the server receives statistical data associated with at least one characteristic of the patient. In some embodiments, the server receives data from the wearable device via wireless communication, wherein communication between the server and the wearable device is carried out via a proxy gateway. In some embodiments, the server may be embedded on the wearable device.

In some embodiments, the dedicated deep learning algorithm includes at least one of: convolutional neural network (CNN), long short-term memory networks (LSTM), and a fully connected neural network model. In some embodiments, the dedicated deep learning algorithm is trained with self-supervision learning.

In some embodiments, the first metric includes at least one of: walk duration, activity intensity level (e.g. calculated using the variability of at least one of the signals or inputs such as acceleration, heart activity, oxygen saturation, time of day (TOD) metrics, and wakeup-dependent metrics), time of day (TOD) metrics, and wakeup-dependent metrics. In some embodiments, the second metric includes at least one of: a gait score, an activity patterns score, an overall performance score, and a cognitive approximation score.

In some embodiments, the combined approximation is normalized to reflect a specific neurologic disorder, and wherein the neurologic disorder is at least one of: Multiple Sclerosis (MS), Parkinson Disease (PD), and Dementia. In some embodiments, the patient's performance is monitored over time to identify events including at least one of: fall events, pain, fatigue, and a spasm.

In some embodiments, the server receives gait speed data from at least one sensor, and wherein the 3D acceleration data is acquired during the gait speed measurement by the at least one sensor, and determines correlation between the patient's gait speed and the received 3D acceleration data.

In some embodiments, the at least one sensor includes a pressure sensor that is embedded in at least one of: a pressure mat and an insole, and wherein the pressure sensor measures pressure that is caused by the patient stepping on the pressure sensor. In some embodiments, the server determines a walking stage by the patient as an indication that the patient is no longer lying in bed, wherein the walking stage is determined by combining the received 3D acceleration data and the at least one physiological signal.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features and advantages thereof, may best be understood by reference to the following detailed description when read with the accompanied drawings. Embodiments of the invention are illustrated by way of example and not limitation in the figures of the accompanying drawings, in which like reference numerals indicate corresponding, analogous or similar elements, and in which:

FIG. 1 shows a block diagram of an example computing device, according to some embodiments of the invention;

FIGS. 2A-2B show a schematic block diagram of a system for progression assessment of a neurological disease of a patient, according to some embodiments of the invention;

FIG. 3 shows a schematic block diagram of a system for progression assessment of a neurological disease of a patient based on gait speed measurement, according to some embodiments of the invention;

FIGS. 4A-4B show a flowchart for a method of estimating progression of a neurological disease of the patient, according to some embodiments of the invention;

FIG. 5 shows a flowchart for a method of progression assessment of a neurological disease of the patient, according to some embodiments of the invention;

FIG. 6 depicts an example raw signal segmentation and feature hierarchy, according to some embodiments of the invention; and

FIG. 7 depicts an example segmentation level, according to some embodiments of the invention.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE INVENTION

In the following detailed description, numerous specific details are set forth in order to provide a thorough understanding of the invention. However, it will be understood by those skilled in the art that the present invention may be practiced without these specific details. In other instances, well-known methods, procedures, and components, modules, units and/or circuits have not been described in detail so as not to obscure the invention. Some features or elements described with respect to one embodiment may be combined with features or elements described with respect to other embodiments. For the sake of clarity, discussion of same or similar features or elements may not be repeated.

Although embodiments of the invention are not limited in this regard, discussions utilizing terms such as, for example, “processing”, “computing”, “calculating”, “determining”, “establishing”, “analyzing”, “checking”, or the like, may refer to operation(s) and/or process(es) of a computer, a computing platform, a computing system, or other electronic computing device, that manipulates and/or transforms data represented as physical (e.g., electronic) quantities within the computer's registers and/or memories into other data similarly represented as physical quantities within the computer's registers and/or memories or other information non-transitory storage medium that may store instructions to perform operations and/or processes. Although embodiments of the invention are not limited in this regard, the terms “plurality” and “a plurality” as used herein may include, for example, “multiple” or “two or more”. The terms “plurality” or “a plurality” may be used throughout the specification to describe two or more components, devices, elements, units, parameters, or the like. The term set when used herein may include one or more items. Unless explicitly stated, the method embodiments described herein are not constrained to a particular order or sequence. Additionally, some of the described method embodiments or elements thereof may occur or be performed simultaneously, at the same point in time, or concurrently.

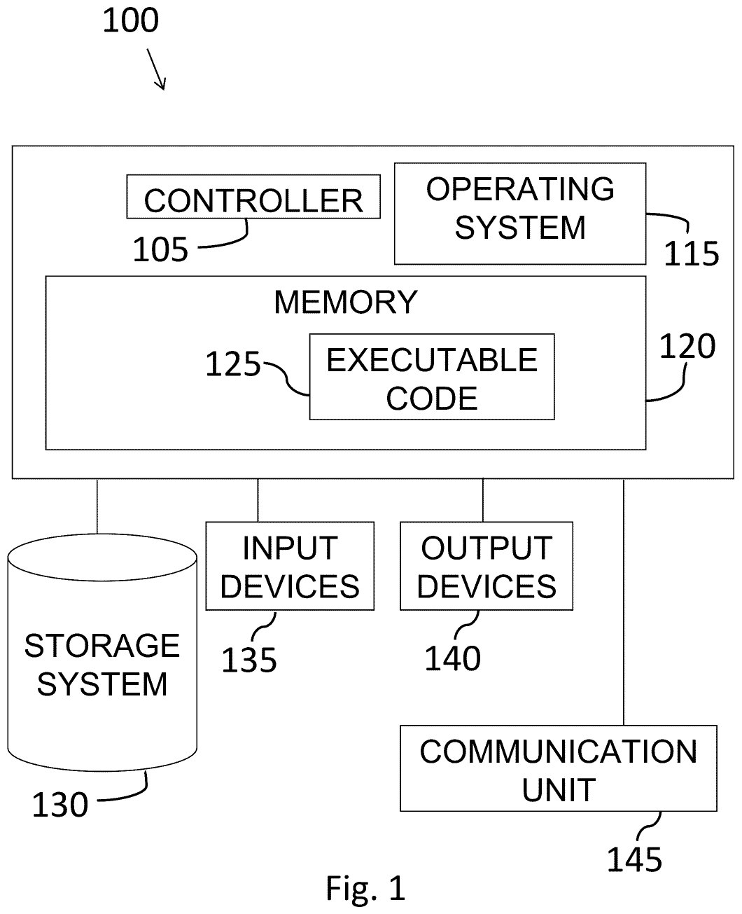

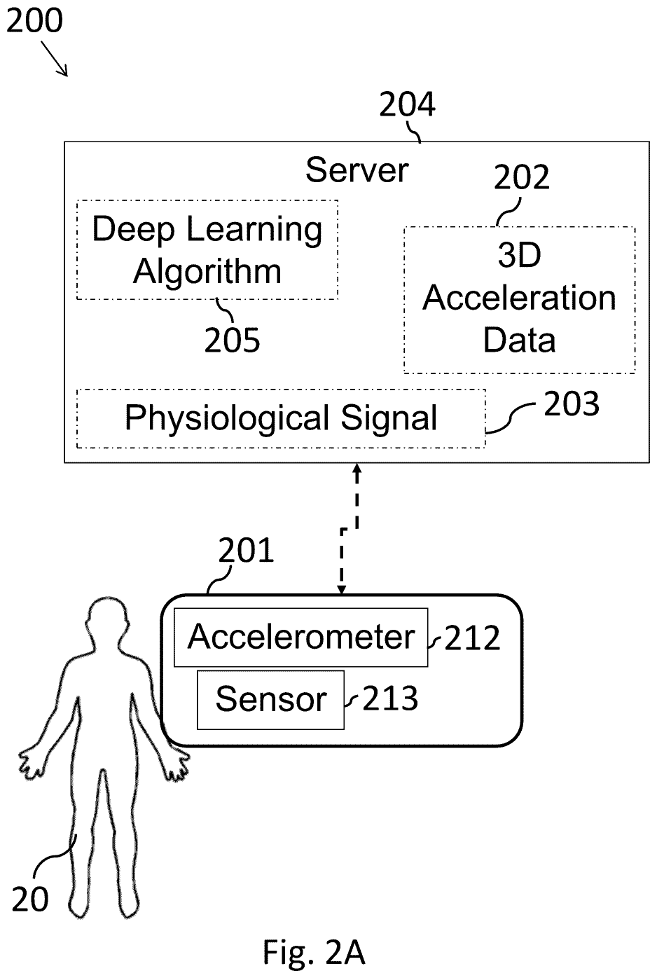

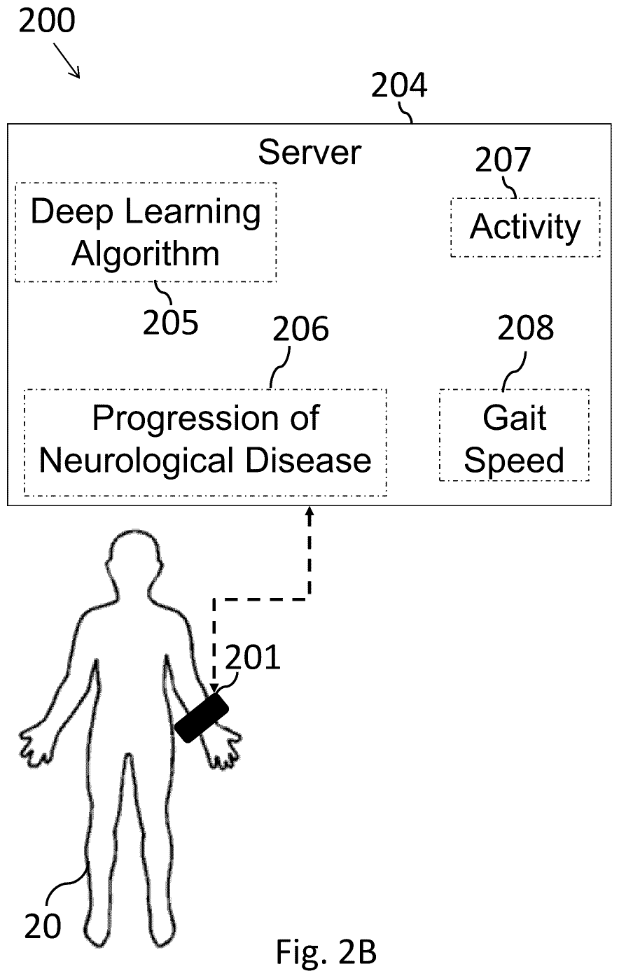

Reference is made to FIG. 1, which is a schematic block diagram of an example computing device, according to some embodiments of the invention. Computing device 100 may include a controller or processor 105 (e.g., a central processing unit processor (CPU), a chip or any suitable computing or computational device), an operating system 115, memory 120, executable code 125, storage 130, input devices 135 (e.g. a keyboard or touchscreen), and output devices 140 (e.g., a display), a communication unit 145 (e.g., a cellular transmitter or modem, a Wi-Fi communication unit, or the like) for communicating with remote devices via a communication network, such as, for example, the Internet. Controller 105 may be configured to execute program code to perform operations described herein. Embodiments may include one or more computing device(s) 100, for example, to act as the various devices or the components shown in FIGS. 2A-2B. For example, components of system 200 may be, or may include computing device 100 or components thereof.

Operating system 115 may be or may include any code segment (e.g., one similar to executable code 125 described herein) designed and/or configured to perform tasks involving coordinating, scheduling, arbitrating, supervising, controlling or otherwise managing operation of computing device 100, for example, scheduling execution of software programs or enabling software programs or other modules or units to communicate.

Memory 120 may be or may include, for example, a Random Access Memory (RAM), a read only memory (ROM), a Dynamic RAM (DRAM), a Synchronous DRAM (SD-RAM), a double data rate (DDR) memory chip, a Flash memory, a volatile memory, a non-volatile memory, a cache memory, a buffer, a short term memory unit, a long term memory unit, or other suitable memory units or storage units. Memory 120 may be or may include a plurality of similar and/or different memory units. Memory 120 may be a computer or processor non-transitory readable medium, or a computer non-transitory storage medium, e.g., a RAM.

Executable code 125 may be any executable code, e.g., an application, a program, a process, task or script. Executable code 125 may be executed by controller 105 possibly under control of operating system 115. For example, executable code 125 may be a software application that performs methods as further described herein. Although, for the sake of clarity, a single item of executable code 125 is shown in FIG. 1, a system according to embodiments of the invention may include a plurality of executable code segments similar to executable code 125 that may be stored into memory 120 and cause controller 105 to carry out methods described herein.

Storage 130 may be or may include, for example, a hard disk drive, a universal serial bus (USB) device or other suitable removable and/or fixed storage unit. In some embodiments, some of the components shown in FIG. 1 may be omitted. For example, memory 120 may be a non-volatile memory having the storage capacity of storage 130. Accordingly, although shown as a separate component, storage 130 may be embedded or included in memory 120.

Input devices 135 may be or may include a keyboard, a touch screen or pad, one or more sensors or any other or additional suitable input device. Any suitable number of input devices 135 may be operatively connected to computing device 100. Output devices 140 may include one or more displays or monitors and/or any other suitable output devices. Any suitable number of output devices 140 may be operatively connected to computing device 100. Any applicable input/output (I/O) devices may be connected to computing device 100 as shown by blocks 135 and 140. For example, a wired or wireless network interface card (NIC), a universal serial bus (USB) device or external hard drive may be included in input devices 135 and/or output devices 140.

Embodiments of the invention may include an article such as a computer or processor non-transitory readable medium, or a computer or processor non-transitory storage medium, such as for example a memory, a disk drive, or a USB flash memory, encoding, including or storing instructions, e.g., computer-executable instructions, which, when executed by a processor or controller, carry out methods disclosed herein. For example, an article may include a storage medium such as memory 120, computer-executable instructions such as executable code 125 and a controller such as controller 105. Such a non-transitory computer readable medium may be for example a memory, a disk drive, or a USB flash memory, encoding, including or storing instructions, e.g., computer-executable instructions, which when executed by a processor or controller, carry out methods disclosed herein. The storage medium may include, but is not limited to, any type of disk including, semiconductor devices such as read-only memories (ROMs) and/or random-access memories (RAMs), flash memories, electrically erasable programmable read-only memories (EEPROMs) or any type of media suitable for storing electronic instructions, including programmable storage devices. For example, in some embodiments, memory 120 is a non-transitory machine-readable medium.

A system according to embodiments of the invention may include components such as, but not limited to, a plurality of central processing units (CPUs), a plurality of graphics processing units (GPUs), or any other suitable multi-purpose or specific processors or controllers (e.g., controllers similar to controller 105), a plurality of input units, a plurality of output units, a plurality of memory units, and a plurality of storage units. An embodiment may additionally include other suitable hardware components and/or software components. Some embodiments may include or may be, for example, a personal computer, a desktop computer, a laptop computer, a workstation, a server computer, a network device, or any other suitable computing device. For example, a system as described herein may include one or more facility computing device 100 and one or more remote server computers in active communication with one or more facility computing device 100 such as computing device 100, and in active communication with one or more portable or mobile devices such as smartphones, tablets and the like.

Reference is made to FIGS. 2A-2B, which show a schematic block diagram of a system 200 for progression assessment (e.g., continuous ongoing and long term) of a neurological disease of a patient 20, according to some embodiments of the invention. In FIGS. 2A-2B, hardware elements are indicated with a solid line and the direction of arrows indicate a direction of information flow between the hardware elements.

The patient 20 may wear at least one wearable device 201 (e.g., a smartwatch) to monitor the patient 20. In some embodiments, wearable device 201 is configured to detect three-dimensional (3D) acceleration data 202 (e.g., from an accelerometer 212) and/or at least one physiological signal 203 (e.g., from a dedicated sensor 213) of the patient 20. An embodiment of the system 200 may provide long-term and/or continuous monitoring of the patient 20 based on data measured by wearable device 201.

Wearable device 201 may be for instance a standard smart watch that allows for ongoing23/7 monitoring (excluding about one hour daily for charging). For example, the wearable device 201 may be designed to be water-resistant, so that patients may wear it during activities such as swimming or showering. System 200 may be Health Insurance Portability and Accountability Act (HIPPA) and/or General Data Protection Regulation (GDPR) compliant, meaning that the data is secured, and personal identifying data is stored separately from sensor data.

For example, the physiological signal 203 may include oxygen saturation data with SPO2 measurements. In another example, the physiological signal 203 may include information on heart activity such as heart rate data, and/or heart rate variability data, and/or a lead electrocardiogram (ECG).

System 200 may include a server 204 (e.g., such as computing device 100, shown in FIG. 1). The server 204 be in communication with the wearable device 201, for instance via wired or wireless communication (e.g., via Bluetooth). The communication may also be via a proxy server, for instance communicating via a Wi-Fi network to a home router, and then from the router to the destination via the Internet.

Server 204 may store the raw sensor data from the at least one wearable device 201 in cloud storage, and save statistical aggregations of the data, including counts, summation, standard deviations, quantiles, skewness, kurtosis, and rolling measures within the past few hours, past day, past week, past month, and for the entire patient history.

Server 204 may be configured to receive at least one of: the 3D acceleration data 202 and/or at least one physiological signal 203 from the wearable device 201. For example, the server 204 may receive statistical data associated with at least one characteristic of the patient 20.

In some embodiments, the 3D acceleration data 202 is sampled with a frequency of at least 25 Hz.

According to some embodiments, system 200 may identify the progression of a neurological disease of the patient 20 over time by modeling signals received from the wearable device 201. Instead of only evaluating a patient's disease state during infrequent visits to a laboratory or clinic, the wearable device 201 may continuously or periodically collect data on the patient's symptoms and movements, thereby allowing for a more comprehensive understanding of the disease progression.

In some embodiments, the data collected from the wearable device 201 may also be used to evaluate the risk of deterioration and/or measure the progression of a neurological disease. This information may be used to make more conscious and timely decisions about changing a patient's medication, postponing or advancing the next clinic visit, or taking other actions to manage the patient's condition thereby allowing for more personalized treatment plans and better disease management.

The server 204 may be configured to apply a dedicated deep learning algorithm 205 to determine a progression of a neurological disease 206 of the patient 20. In some embodiments, the dedicated deep learning algorithm 205 is trained to receive 3D acceleration data 202, e.g. data regarding acceleration in three different directions, such as X, Y and Z each perpendicular to each other, and/or at least one physiological signal 203 as input, and accordingly provide analysis on progression of the neurological disease 206 (e.g., inferred from the dedicated deep learning algorithm 205). Additional data other than 3D acceleration data may be used.

The dedicated deep learning algorithm 205 may be trained separately for different types of neurological diseases, such as Parkinson's disease or Multiple Sclerosis (MS).

According to some embodiments, determination of the progression of the neurological disease 206 includes aggregation of the patient's 20 characteristics. For instance, characteristics such as number of steps walked by the patient, velocity of the patient at a given time, heart rate at a given time, and the like. A first metric may be determined for patient's activity 207, a second metric may be determined based on a measured gait speed 208 and the first metric and the second metric may be aggregated into a combined approximation and/or inference for progression of the neurological disease 206.

In some embodiments, different characteristics of patient 20 have different weights such that mapping of the aggregated metrics may correspond to the inference for progression of the neurological disease 206 (e.g., calculated in parallel to inference from the 3D acceleration data 202). The inference for progression of the neurological disease 206 may be calculated more accurately based on the aggregated metrics when compared to calculation based on the 3D acceleration data 202.

According to some embodiments, server 204 employs computational models to track the advancement of neurological disease 206. These models may be structured in a three-tiered architecture, which may add diverse learning sub-objectives during the algorithm optimization phase, that supplements additional information that is assessable individually, yet provides value when amalgamated. The first tier encompasses basic metrics related to the patient's activities 207 such as duration of walking, intensity of activity, time-of-day measurements, and metrics dependent on waking up. These metrics may be extracted using signal processing and aggregation of simple extracted metrics. The next tier may include more intricate metrics, involving assessed gait speed 208, gait scoring, activity pattern scoring, comprehensive performance scoring, and cognitive estimation scoring these metrics are learned from the signals of the at least two wearable sensors by aligning their signal with acquired labels in lab conditions and outside of the lab (e.g. real world conditions). The concluding tier integrates the scores from the lower tiers to form a collective estimation of disease progression, which could be standardized to align with the typical scale of the specific neurological condition. These tiers or layers could either function as key expected outputs of a single, unified neural network or could be the results of various separate neural networks, which are trained individually per each or some of the targets of layers 2-3, and later combined post-training along with their trained weights followed by additional training of some added layers for the downstream task. server 204 may utilize algorithmic models to assess the progression of the neurological disease 206, which are divided into three layers. These three layers serve as multiple training targets for the algorithm fine-tuning stage, each adding some information that can be measured separately but when combined create a robust and solid assessment of continuous progression scoring. The first layer may include low-level metrics for patient's activity 207 such as walk duration, activity intensity level, time of day (TOD) metrics, and wakeup-dependent metrics. The second layer includes metrics of higher complexity, including a measured gait speed 208, gait score, activity patterns score, overall performance score, and cognitive approximation score. The final layer aggregates the lower-level scores into a combined approximation of the disease progression, which may be normalized to reflect the standard scale of the specific neurologic disorder (e.g., EDSS for MS, UPDRS for Parkinson's, and CDR for dementia).

The dedicated deep learning algorithm 205 may include at least one of: convolutional neural network (CNN), long short-term memory networks (LSTM), and a fully connected neural network model. The training mechanism may include self-supervision phase followed by a fine-tuning phase to fit to each of the endpoints described. For example, an initial phase of fitting the deep neural network may be made over the entirety of the available data both in the clinic and outside of it with the target to identify common and or significant patterns within the signal itself, once trained this allows for a better mapping between the data from the at least two sensors and any or all of the layers described elsewhere herein metrics may be initially developed in a controlled environment (e.g., a dedicated gait and cognition laboratory) and later adapted for usage within daily living environments.

An algorithm and a system may use a series of neural network layers to process the sensor data. An input layer of a NN may receive sensor data from e.g. a wristwatch, for example acceleration data, physiological signals, heart data, oxygen data, etc. Base sub-models for various metrics may include physiological estimators (such as sedentary behavior, sleeping, activity level, physical effort, etc.) and aggregations of the raw signal (such as number of spikes in the raw signal, mean of signal per each channel of 3D data (x,y,z), maximum value of the heart rate, etc.). A sub-model may include a gait speed regressor that uses a separate deep neural network that was trained to estimate the gait speed, for example using laboratory and outdoor collected acceleration and heartrate data, and which may be validated, e.g. using insoles, back worn and mat sensors. A sub-model may include a walk duration or step count for instance specifically designed and or trained for at-risk populations (e.g., elderly, neurological disease patients, with and without walking aids), e.g. as disclosed in U.S. Patent Publication No. 2023/0081657, incorporated by reference herein. A sub-model may include a standard deviation of 3D acceleration data over a period of data (e.g. 5 seconds of data) with a specific stride (e.g., a stride of 1 second between aggregation). A sub-model may include a k-means clustering of the standard deviation of data such as acceleration data, to form different clusters of activity patterns during the data. A sub-model may include a k-means clustering of the standard deviation of acceleration data to form different clusters of patients by their activity patterns. A sub-model may include a sleep duration estimation, for example measured by time accumulation of sequences in which movement and heart rate were in certain ranges, e.g. under their median values during the past week respectively. For example, accumulation of such data may be done per each 24 hour period during a certain time period, e.g. 6:30 AM-6:30 PM. A sub-model may include a sleep interruptions model, for example defined as non-sleep sequences between sleep sequences, for example sleep duration sequences as described herein. A sub-model may include a first morning rise model, e.g. defined as the timing and time measured between first non-sleep sequence as in a sleep interruptions model described herein, that is not followed by a sleep sequence (e.g. for the following hour) and the first walking sequence as described herein. A sub-model may include statistical measures of heart rate variability, heart rate, acceleration; a statistical aggregations function may include at least one of: minimum, mean, max, 10th percentile, 90th percentile, standard deviation, or other statistical functions. A sub-model may include age or gender metadata. According to some embodiments, aggregation may include at least one of the following: an aggregation of a specific indicator over time (e.g., the standard deviation of raw acceleration data over a period of 5 minutes), an aggregation of a specific measure over time of day (TOD) fragments of a period (e.g., median heart rate calculated on data from 7a.m. to 11a.m. over a week time).

For example, aggregation of multiple inputs may include, for example, computing a weighted sum of the inputs (e.g. gait speed*0.4+sleep duration*0.1+ (heart_rate_minimum>130)*0.5); aggregating by using a learnable mechanism of Feed Forward Neural Network for the inputs (e.g. every layer gradually converges to the optimal weight per each input and desired output on predefined training set so the layer aggregates the inputs, and several layers can be stacked one on top of the other such that all layer outputs serves as the next layer inputs). Aggregating inputs may use a nonlinear function (e.g. y=x if x>0, else y=0) to enable a growing complexity representation and hence allow for a better mapping from the inputs to our desired metric (as stacking of linear functions alone can be reduced to a single linear function of the inputs).

For example, the aggregation may include calculation of a linear function ((0.229*input a +0.457*input b+0.9238*input c)>0.7)*92839, with some statistical function over the inputs (e.g. calculating maximum of [standard deviation (hr), standard deviation (gait speed)*100, standard deviation (activity intensity)*25], or for a mixture of the previous reported aggregations.

For example, to further demonstrate some relevant layers' usage including their inputs and the flow between them, the following example process may be used for ongoing assessment of MS progression. The first layer inputs may be: (a) activity intensity measured by standard deviation of raw acceleration data over 1 minute cycles with aggregation for monthly, weekly, daily, hourly and 4 daily time fragments of 6 hours starting 5a.m. (b) high intensity activity duration (e.g., for std>3.2 with the same 1 min cycles std described in (a)) that are aggregated (e.g., for count of minutes) monthly, weekly, daily and hourly per each hour for the first 5 hours following daily first rise from bed. (c) min, average, median max and skew statistic calculation of heart rate, each measured on 96 fragments of 15 minute duration starting 5a.m. for the current day (d) step counting e.g. as disclosed in U.S. Patent Publication No. 2023/0081657, incorporated by reference herein, aggregated daily, hourly and 4 daily time fragments of 6 hours starting 5a.m. (e) counting of number of deviations from personal historical measurement statistics: daily number of times heart rate exceeded personal daily 80^{th} percentile references calculated on past week and on past month. (f) number of night wakeups per each night of the past week. (g) a ratio calculated as the division between daily metrics and daily average of the same metrics during the past week and during the past month. The metrics for these ratios are: daily step count daily percentile of time with activity intensity >2, and with activity intensity >3.2, daily 80^{th}, 90^{th} and 95^{th} percentile of heart rate. All these scalar metrics are then concatenated to a single vector, are normalized across the entire training data vector (feature-wise) and serve as the first input of the artificial neural network.

The second layer inputs may be: (a) gait speed assessed as further described hereinafter, for which perform calculation of min, max, average, 90th and 10th percentiles for 76 fragments of 15 minutes each measured from 5a.m. to midnight. (b) gait speed assessed for which we calculate min, max, average, 90th and 10th percentiles over a period of day, week, and month. (c) the ratio between the above daily statistics of gait speed divided by the monthly stats of gait speed. (d) gait regularity assessment for which a process may calculate min, max and median on a daily aggregate level and monthly mean of these daily measurements. (e) binary indicator for change larger than 0.2 in daily gait regularity relatively to the monthly mean. (f) cognitive estimates measured as weekly average and 90th percentile of response time to a watch transmitted indication. These metrics may be divided by monthly average of the same measurements. All these scalar metrics may then be concatenated to a single vector, normalized across the entire training data vector (feature-wise) and serve as the second input of ML model, e.g. the artificial neural network.

The third layer inputs are the outputs of the previous two layers that are fed to a fully connected (dense) neural network, along with the raw signals for acceleration data (at 25 hz), heart rate data (at 1 hz) and heart rate variability data (at 1 hz), each inserted to the network twice (1) into an LSTM block and (2) into a 1D convolutional block. The outputs of all these input receiving blocks are then concatenated along with the outputs of the fully connected layers and are processed by additional layers together.

The final output of this architecture is a score assessing the progression of MS which may then be scaled to match the clinically accepted EDSS scale. Other scales may be also used (e.g., for other conditions). In this specific described case the part of the architecture that receives the raw acceleration, heart rate and heart rate variability data is pre-trained prior to the above described training process using partial masking of the inputs and a reconstruction target and loss. Once pre-training phase is completed the reconstruction heads (layers outputting the assessed original unmasked inputs) are removed and weights achieved in this process are used for a good initialization of this pretrained part of the overall network.

A NN in one embodiment may include a series of convolutional layers used to extract relevant temporal features from the sub-models' data. Additional pooling layers may be used between convolutional layers and may be applied to reduce the sequence (e.g. temporal) dimension of the signal while retaining crucial information. A series of fully connected layers may be used over the one or more of the outputs of sub-models for classification purposes, determining the presence or absence of an event, and/or regression tasks, estimating the extent of a condition or a non-discrete measure based on the extracted features. NN output layers may predict the progression level of the neurological disease.

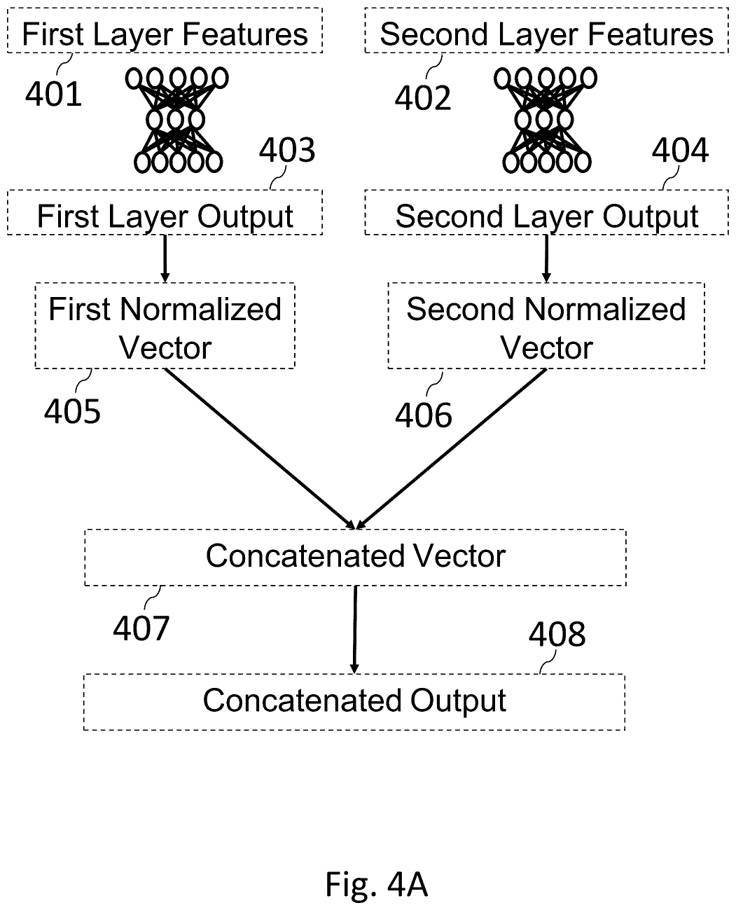

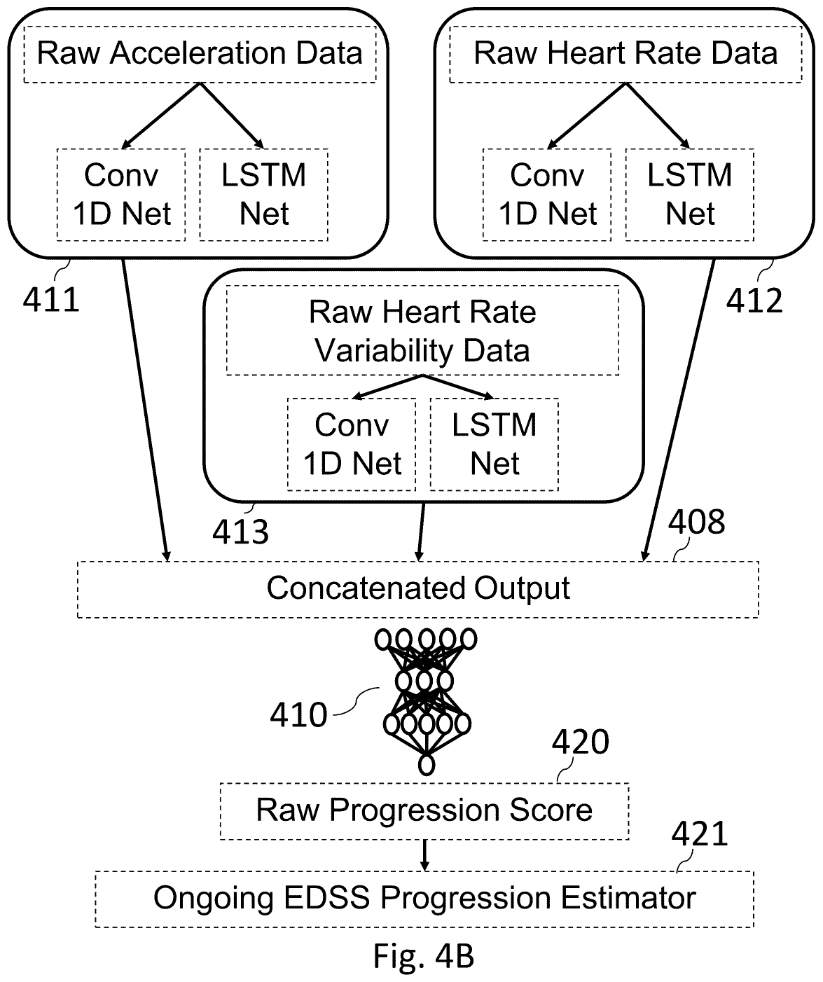

Reference is made to FIGS. 4A-4B, which show a flowchart for a method of estimating progression of a neurological disease of the patient, according to some embodiments of the invention.

In order to estimate progression of a neurological disease of the patient, an artificial neural network 410 may receive input from several layers. A first layer may handle simple features which are not modeled (e.g., signals related to a subject's gait). The first layer features 401 may be inputted to a dedicated neural network to receive a first layer output 403 and normalized to a first normalized vector 405. The first normalized vector 405 may be fed as one of the inputs for the artificial neural network 410.

Similarly, a second layer may use pre-trained features to calculate gait speed and/or gait regularity. The second layer features 402 may be input to a dedicated neural network to receive a second layer output 404 and normalized to a second normalized vector 406. The second normalized vector 406 may be fed as one of the inputs for the artificial neural network 410.

The first normalized vector 405 and the second normalized vector 406 may be concatenated to a concatenated vector 407 and aggregated to a concatenated output 408 with other inputs.

The concatenated output 408 may also receive as input raw acceleration data 411, that is in turn fed to a 1D CNN (convolutional) layer or block of a NN and an LSTM block. The concatenated output 408 may also receive as input raw heart rate data 412 that is in turn fed to a 1D CNN (convolutional) layer or block of a NN and an LSTM block. The concatenated output 408 may also receive as input raw heart rate variability data 413 that is in turn fed to a 1D CNN (convolutional) layer or block of a NN and an LSTM block.

The concatenated output 408 may be input to artificial neural network 410 so that the output is a raw progression score 420. The raw progression score 420 may be adjusted to the EDSS scale (for MS, or other scales accordingly) so that an ongoing EDSS progression estimator 421 may be achieved.

Inputs to a system may be described with respect to three different “layers” which are not to be confused with the layers of NNs used with embodiments of the present invention. A first layer may handle simple features which are not modeled; for example, a first layer may input base measurements or signals related to a subject's gait, and other features. Aggregation in a first layer may determine the number of times (e.g., per period of time) a deviation of a measure such as gait speed or heart rate is larger than 1.5 or another threshold of standard deviations. Such aggregation may detect spikes in measures, and determine the number of such spikes per period of time. Aggregation in a first level may normalize data such as number of steps to certain populations, e.g. normalization of number of steps to people with a certain medical condition. Aggregation, at any layer or level, may function to combine a number of different measures.

A second layer may use externally trained model, to handle, e.g. granularity of walking, regularity of walking, gait speed etc. A second layer may use a pre-trained model (e.g. a NN or other model) to calculate gait speed and/or gait regularity.

Each of the first, second and third layers may perform aggregation in different manners. For example, the first layer may perform an average of standard deviations, counts, etc. Each of the first, second and third layers may output in the form of, for example, a vector (e.g. an ordered list of numbers).

The output of first and second layers may be input to a third layer. The third layer may accept input such as first layer output and second layer outputs (each of which may be a vector) and in addition other data such as the raw patient data.

The third layer may be a ML model, such as a NN, which may accept data in the form of a, for example, a tensor (e.g. a 4D or 5D tensor). The third layer may be a NN accepting the first and second layer vector outputs and in addition signals such as a raw data signal of acceleration at, e.g. 25 hz, and a raw signal of 1 hz heartrate variability and raw heartrate, and input these signals to specific sections of a NN. Raw data may be input as a tensor such as a 4D tensor. For example, signals in a third layer may be input to an LSTM block, and/or a ID CNN (convolutional) layer or block of a NN. The NN may output a vector.

Output of the three-layer process may describe a progression of a disease. Typical output may be a standard description of the progression of a disease according to known standards for describing that disease. E.g., a rating on the standard scale for progression of Parkinson's disease may be output.

In order to use system 200, the patient 20 may use a dedicated mobile app (to be executed on a mobile device such as a smartphone in communication with the wearable device 201) that allows patients and/or caregivers to monitor the patient's performance over time, tag special events such as fall events, pain, fatigue, etc., and monitor trends of all these metrics and reported events over time. This may assist the patient or caregiver to manage the neurological disease and be more data-driven when meeting with the clinician. A corresponding clinician terminal may be provided for full access to all levels of metrics, along with a triage table that weighs both the absolute score of each patient and the personal change in score over the past days.

According to some embodiments, additional metrics may be used for determination of the progression of the neurological disease 206. Some base metrics for at least one of: gait speed and activity fragmentation, may be calculated specifically over the time frame adjacent to the morning first rise (when the patient stands up from a sleeping state). This period may be critical in assessing the overall patient health and disease progression. During this time, the patient's body transitions from a resting state to an active one and measuring these metrics provides valuable insights into the individual's physical capacity and functional independence.

For instance, low activity intensity or slower gait speeds may indicate mobility limitations or early signs of neurological disorders, while high activity fragmentation may suggest poor sleep quality or musculoskeletal issues. By adding these metrics, it may be possible to promptly identify subtle changes in patient's health, enabling early intervention and personalized care plans, improving disease management and enhancing the overall quality of life.

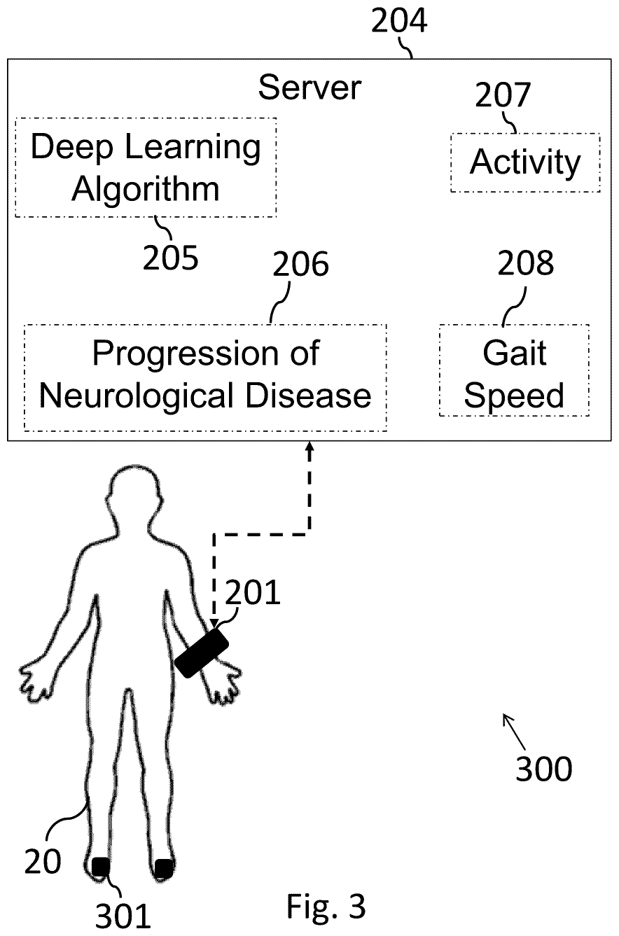

Reference is made to FIG. 3, which shows a schematic block diagram of a system 300 for progression assessment of a neurological disease of a patient 20 based on gait speed 208 measurement, according to some embodiments of the invention.

According to some embodiments, the gait speed 208 is accurately measured by at least one sensor 301. The 3D acceleration data, indoor and outdoor without GPS data (e.g., as shown in FIG. 2A) may be accordingly acquired during the gait speed measurement by the at least one sensor 301. For example, elderly people usually spend most of their time indoors, or travel small distances during the day, so that a GPS signal is not available.

Thus, the server 204 may determine correlation between the patient's gait speed 208 and the received 3D acceleration data so as to determine a baseline for correct gait speed measurements. In some embodiments, the gait speed may be determined by the server 204 from new 3D acceleration data based on this correlation.

In some embodiments, the at least one sensor 301 may include a pressure sensor that is embedded in at least one of: a pressure mat (e.g., capable of measuring pressure on a 1-centimeter resolution) and/or an insole. The pressure sensor may measure pressure that is caused by the patient 20 stepping on the pressure sensor.

For example, the calculation of the gait speed may be conducted by equipping the patient 20 with two sensors at the same time and fitting the mapping from wearable device 201 sensor data to match the measurements acquired from the pressure sensor.

The gait speed 208 may also be determined by other type of measurements such as a light sensor measuring response time of a dedicated laser beam, and/or using image processing on video data of the patient's movement in a laboratory environment.

According to some embodiments, once trained, gait speed may be inferred for other patients that did not have a reference measurement with a dedicated reference sensors (e.g., a pressure sensor).

In some embodiments, the server may determine a walking stage by the patient as an indication that the patient is no longer lying in bed, where the walking stage is determined by combining the received 3D acceleration data and the at least one physiological signal. Wakeups may be defined as the action of transitioning from sleep to standing/walking. The difficulty arises from multiple possible movements while sleeping ranging from posture changes within bed to tremor or other continuous movements for some of the neurological patients. In addition, the wearable device 201 is not constrained to the center of the patient's body mass thereby reducing the ability to detect the body posture while static.

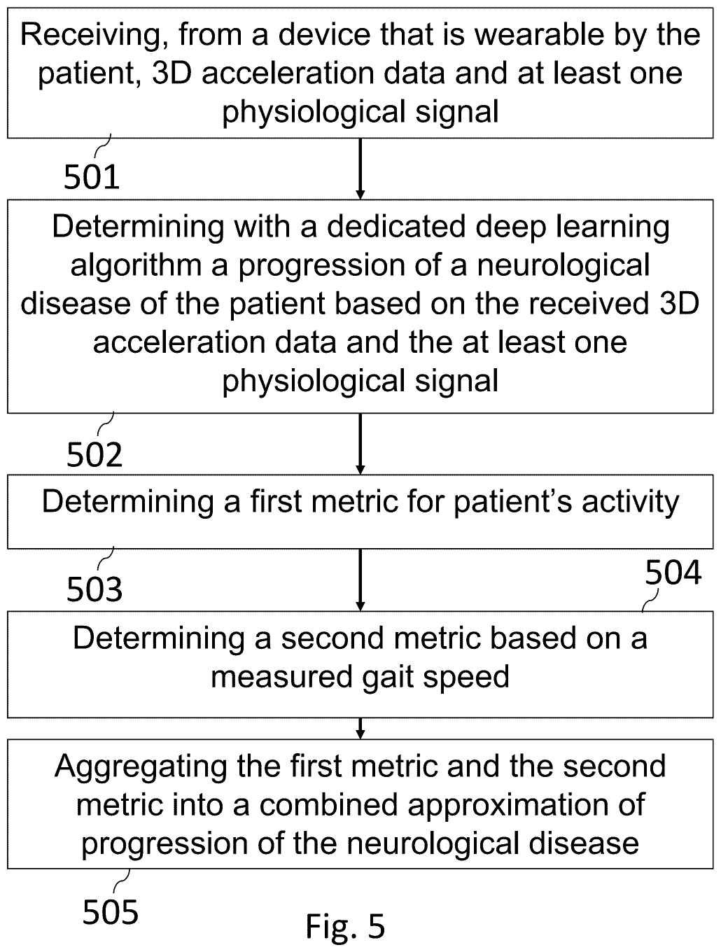

Reference is made to FIG. 5, shows a flowchart for a method of progression assessment of a neurological disease of the patient, according to some embodiments of the invention.

Initially, 3D acceleration data and at least one physiological signal may be received in operation 501 from a device that is wearable by the patient.

The server may determine in operation 502 with a dedicated deep learning algorithm a progression of a neurological disease of the patient based on the received 3D acceleration data and the at least one physiological signal.

The determination of the progression of the neurological disease may include: determining in operation 503 a first metric for patient's activity, determining in operation 504 a second metric based on a measured gait speed, and aggregating in operation 505 the first metric and the second metric into a combined approximation of progression of the neurological disease.

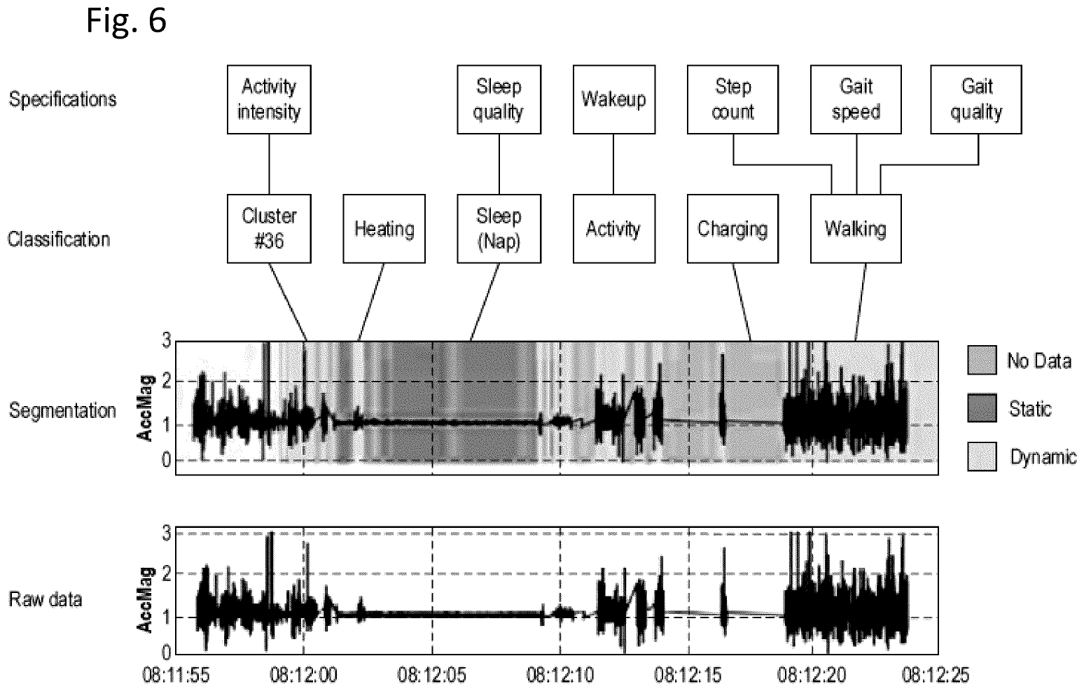

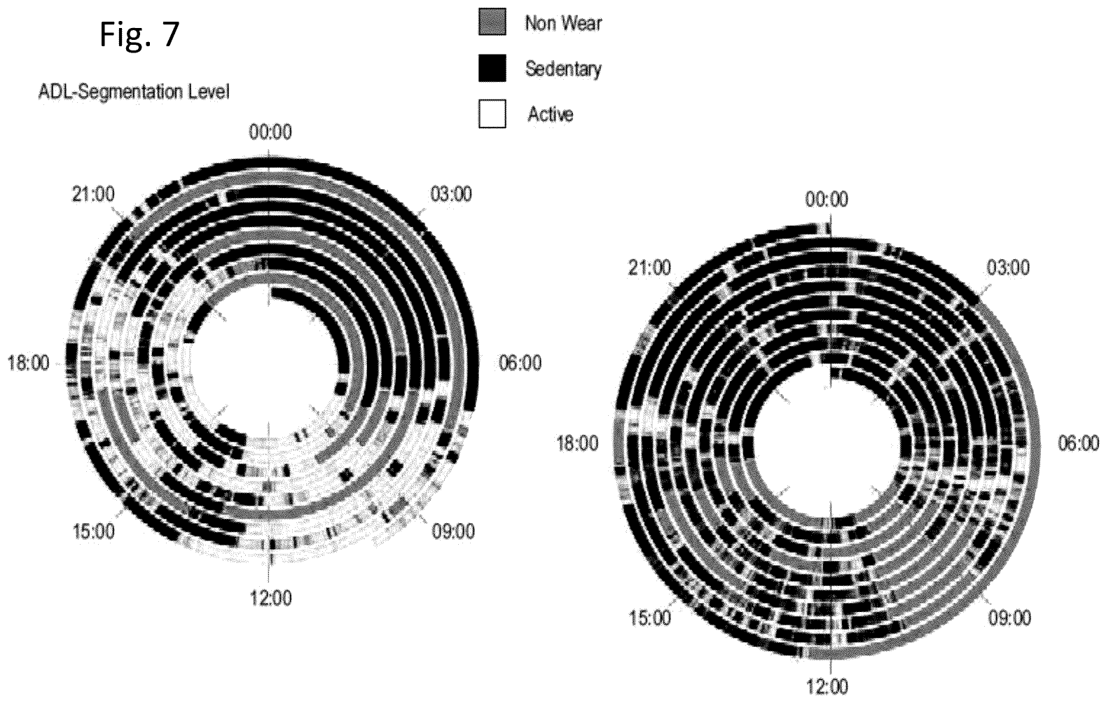

FIG. 6 depicts an example raw signal segmentation and feature hierarchy, according to some embodiments of the invention. The data and processing shown in FIG. 6 may determine a second metric. A raw signal may be processed to assess explained metrics such as active time vs. rest time, type of activity such as walking vs. sitting, and also in order to serve as an additional input in itself towards the overall progression score. FIG. 7 depicts an example segmentation level, according to some embodiments of the invention. FIG. 7 describes a timeline with subject activity (e.g. not wearing device; sedentary, active) correlated with time of day (TOD). The right side of FIG. 7 depicts fixed patterns through the days of the week with stable charge timing and duration with analysis of 9 days which are synced to match the same time of day (TOD) (e.g., north=midnight cast=6 am etc.). Using such presentation of the data may enable easier conversion of the time of day metrics as well as insights to features of the overall progression score, since some conclusions on the activity cannot be otherwise achieved (e.g., a highly active patient with an unusual activity at a particular time of day). The left side of FIG. 7 shows changing patterns of activity and rest time (e.g., varying charge timing and duration of the monitoring device).

The activities of daily living (ADL) may be examined, where FIG. 7 shows the differences between the behavioral patterns as they are expressed in the sensors (and metrics which we derive from the sensors) throughout the day and across the days of the week. Each cycle in the spiral represents a day and the hours of day are always at the same direction in each layer of the spiral (e.g. north is midnight south is midday).

While certain features of the invention have been illustrated and described herein, many modifications, substitutions, changes, and equivalents may occur to those skilled in the art. It is, therefore, to be understood that the appended claims are intended to cover all such modifications and changes as fall within the true spirit of the invention.

Various embodiments have been presented. Each of these embodiments may of course include features from other embodiments presented, and embodiments not specifically described may include various features described herein.

1. A method of progression assessment of a neurological disease of a patient, the method comprising:
receiving, from a device that is wearable by the patient, three-dimensional (3D) acceleration data and at least one physiological signal;
determining, by a server, with a deep learning algorithm a progression of a neurological disease of the patient based on the received 3D acceleration data and the at least one physiological signal, wherein determination of the progression of the neurological disease comprises:
determining a first metric for patient's activity;
determining a second metric based on a measured gait speed; and
aggregating the first metric and the second metric for approximation of progression of the neurological disease based on inference of the deep learning algorithm.

2. The method of claim 1, wherein the 3D acceleration data is sampled with a frequency of at least 25 Hz.

3. The method of claim 1, wherein the at least one physiological signal comprises information on heart activity with at least one of: heart rate data, heart rate variability data and a 1 lead electrocardiogram (ECG).

4. The method of claim 1, wherein the at least one physiological signal comprises oxygen saturation with SPO2 measurements.

5. The method of claim 1, comprising receiving, by the server, statistical data associated with at least one characteristic of the patient.

6. The method of claim 1, comprising receiving, by the server, data from the wearable device via wireless communication, wherein communication between the server and the wearable device is carried out via a proxy gateway.

7. The method of claim 1, comprising training the deep learning algorithm with self-supervision learning.

8. The method of claim 1, wherein the first metric comprises at least one of: walk duration, activity intensity level.

9. The method of claim 1, wherein the second metric comprises at least one of: a gait score, an activity patterns score, an overall performance score, and a cognitive approximation score.

10. The method of claim 1, wherein the combined approximation is normalized to reflect a specific neurologic disorder, and wherein the neurologic disorder is at least one of:
Multiple Sclerosis (MS), Parkinson Disease (PD), and Dementia.

11. The method of claim 1, comprising monitoring the patient's performance over time to identify events comprising at least one of: fall events, pain, fatigue, and a spasm.

12. The method of claim 1, comprising:
receiving, by the server, gait speed data from at least one sensor, and wherein the 3D acceleration data is acquired during the gait speed measurement by the at least one sensor; and
determining, by the server, correlation between the patient's gait speed and the received 3D acceleration data.

13. The method of claim 1, wherein the at least one sensor comprises a pressure sensor that is embedded in at least one of: a pressure mat and an insole, and wherein the pressure sensor measures pressure that is caused by the patient stepping on the pressure sensor.

14. The method of claim 1, comprising:
determining, by the server, a walking stage by the patient as an indication that the patient is no longer lying in bed, wherein the walking stage is determined by combining the received 3D acceleration data and the at least one physiological signal.

15. The method of claim 14, wherein the at least one physiological signal comprises information on heart activity with at least one of: heart rate data, heart rate variability data and a lead electrocardiogram (ECG).

16. The method of claim 14, wherein the at least one physiological signal comprises oxygen saturation with SPO2 measurements.

17. The method of claim 14, wherein the 3D acceleration data is sampled with a frequency of at least 25 Hz.

18. A system for progression assessment of a neurological disease of a patient, the system comprising:
a wearable device, to monitor three-dimensional (3D) acceleration data and at least one physiological signal; and
a server, in communication with the wearable device, wherein the server is configured to:
receive the 3D acceleration data and the at least one physiological signal; and
determine using a deep learning algorithm a progression of a neurological disease of the patient based on the received 3D acceleration data and the at least one physiological signal, wherein determination of the progression of the neurological disease by the server comprises:
determination of a first metric for patient's activity;
determination of a second metric based on a measured gait speed; and
aggregation of the first metric and the second metric for approximation of progression of the neurological disease based on inference of the deep learning algorithm.

19. The system of claim 18, wherein the combined approximation is normalized to reflect a specific neurologic disorder, and wherein the neurologic disorder is at least one of:
Multiple Sclerosis (MS), Parkinson Disease (PD), and Dementia.
